(19) United States
(12) Patent Application Publication
(10) Pub. No.: US 2013/0259741 A1
(43) Pub. Date: Oct. 3, 2013

(54) MULTI-STAGE NON-THERMAL PLASMA APPARATUS AND METHOD FOR TREATING FLUID FLOWS

(71) Applicant: AIR PHASER ENVIRONMENTAL LTD., Surrey (CA)

(72) Inventor: Douglas Philip Lanz, Surrey (CA)

(73) Assignee: AIR PHASER ENVIRONMENTAL LTD., Surrey (CA)

(21) Appl. No.: 13/846,415

(22) Filed: Mar. 18, 2013

Related U.S. Application Data
(60) Provisional application No. 61/618,492, filed on Mar. 30, 2012.

(51) Int. Cl. A61L 9/22

(52) U.S. Cl. CPC A61L 9/22
USPC 422/4; 422/121; 422/119; 422/108

(57) ABSTRACT

A non-thermal plasma (NTP) system treats fluid flows (e.g., air) containing pollutants (e.g., VOCs; particulate) using two or more stages, each operated to produce NTP with a respective frequency targeted toward one or more pollutants. NTP generation cell assemblies or dielectric barrier discharge devices are arranged in stages along a flow path. Parallel flow paths with multiple stages may be employed. NTP fields in successive stages operate at a different frequency, power density and/or waveforms, which are controlled to be a vibrational harmonic of a targeted compound. Power density can be adjusted to only create gaseous ionized species or to incinerate organic airborne, suspended particulate in situ. Automatic fault detection employs a split ground current so that an imbalance indicates a fault. A faulted component is automatically isolated. Automatic re-routing allows operation with only slightly degraded performance.

### FIELD

This disclosure is related to treatment of fluid flow, and in particular treatment of fluid flows such as air containing various contaminants, pollutants or other undesirable components.

### BACKGROUND

In the case of industrial, commercial, residential or other practical applications there are typically numerous contaminants, pollutants or other undesirable components in a fluid flow that are to be treated. The fluid flow may, for instance, be a flow or stream of air containing various compounds, for instance volatile organic compounds (VOCs), which may or may not be odorous, in any combination and concentration. The fluid flow may additionally, or alternatively include suspended, fine organic particulate known as Particulate Material (PMₓₓ) where the xx designates mean size of the particulates. For example, PM₁₀ designates particles that average 10 micrometers in diameters, and was an early concern of scientists, health officials and regulators. Now PM_{2.5} is becoming an increasing concern. It is estimated that PM pollution causes 22,000-52,000 deaths per year in the U.S. (year 2000) and 200,000 deaths per year in Europe. Much of the PM_{2.5} is believe to result from smoke or diesel fuel combustion. Fine organic particulate may also include aerosol droplets. The fluid flow may have other contaminants or pollutants. The contaminants or pollutants may result from a variety of processes, for instance manufacturing, fabrication, and even cooking.

Some such fluid flows have been treated using various techniques. For example, treatment may include various oxidation or reduction techniques, or incineration. Additionally, fine organic particulate, including organic aerosol droplets, can be incinerated.

One technique that has been employed is exposure of the fluid flow to a non-thermal plasma (i.e., a plasma generated substantially via high voltage rather than heat). One such treatment approach employing a non-thermal plasma is described in U.S. Pat. No. 8,105,546.

While various treatment approaches are commercially feasible, it is desirable to achieve high energy efficiency and/or more thorough disassociation or destruction of the contaminants or pollutants than might otherwise be achieved via conventional approaches. It is also desirable to have a system that is scalable to accommodate a large variety of applications, and one which is configurable to address various contingencies.

### BRIEF SUMMARY

Described herein are structures and methods for treatment of fluid flows, such as air containing contaminants or pollutants, such as volatile organic compounds (VOC) which may or may not be odorous, and/or suspended, fine organic particulate including particulate having approximate diameters in the range of 2.5 microns or less, such as smoke. Such contaminants or pollutants may be emissions produced, for example by commercial and/or industrial materials processing. Such contaminants or pollutants may result from food preparation, for example by restaurants or kitchens which may be located close to a residential area.

The structures and methods employ non-thermal plasmas having frequencies targeted at specific compounds. Without being bound by theory, such induces a vibrational frequency in the molecules of the compounds, advantageously leading to dissociation into safe or simpler forms.

To increase energy efficiency and thoroughness of destruction, a treatment system may use multiple stages of treatment, arranged in series along a flow path of the fluid flow stream to be treated. Each stage may include structures to produce a respective non-thermal plasma field, and be controlled to achieve selected physical characteristics of the respective non-thermal plasma field. The non-thermal plasma produced at each stage may, for example, have a respective frequency, power density and/or waveform. These physical characteristics are selected and controlled to target certain contaminants or pollutants in the fluid flow.

Due to physical limitations on energy and air volume that a single non-thermal plasma field can handle, parallel flow paths may be employed, each with its own set of stages arranged in series along the respective flow path. Such may allow a larger volume of fluid (e.g., air) to be handled than might be handled using a single series arrangement. This also may advantageously provide alternative paths for treatment where one or more components of a given flow path malfunctions, is being serviced or is otherwise unavailable.

Thus, the treatment system may include multiple parallel flow paths, each with multiple stages arranged in series along each respective flow path. The fluid can travel through a first stage for exposure to a first non-thermal plasma, then through a second stage for exposure to a second non-thermal plasma. The fluid may travel through subsequent stages (e.g., tertiary) for exposure to subsequent (e.g., tertiary) non-thermal plasmas. Any number of series arranged stages may be used to achieve the desired effect on the air or gas being treated. The total number of parallel paths or parallel non-thermal plasma fields may be a function of a volume to be treated and/or a level of contaminants or pollutants in the volume. All parallel non-thermal plasma fields at any given stage may operate with the same characteristics (e.g., frequency, power density, wave shape). Each successive stage in series may be operated at a respective frequency, power density and/or wave shape, different from that of the other stages. The successive stages are tuned to excite molecular vibration of a targeted compound so the targeted compound preferentially dissociates, if dissociation is the intended effect, or is preferentially activated for a different reaction if that is the desired effect. The power density can be sufficiently diffuse so as to only create gaseous ionized species or can be sufficiently intense to incinerate organic airborne, suspended particulate in situ by the non-thermal plasma field and active electrical micro arcing. The non-thermal plasma my also incinerate aerosol droplets which burn, such as aerosol oils. The power density may be finely adjustable between a high setting for particulate destruction and VOC destruction if desired, and a low setting if a target VOC can be transformed with a low power density setting.

To generate the non-thermal plasma, each stage may include one or more respective electrode assemblies. The electrode assemblies may take the form of dielectric barrier discharge devices. The dielectric barrier discharge devices may each incorporate a plurality of catalytically active electrodes. The dielectric barrier discharge devices develop one or more non-thermal plasma fields so as to create sufficient reactive oxygen species, hydroxyl species and/or other highly ionized molecules and atomic species so as to cause the oxidation and/or reduction and/or the transformation of targeted compounds (e.g., VOCs). The non-thermal plasma field also produces electron bombardment and collisions with the VOC molecules, which aids in the dissociation of the VOCs. This occurs at the same time that the hydroxyl and reactive oxygen species are created. Parallel non-thermal plasma fields may have similar excitation of power including frequency, power density and waveform, while each sequential non-thermal plasma field in series operates at different respective frequencies, power densities and/or waveforms, with the frequency, power density and waveform selected and controlled to be a vibrational harmonic of the compound that is targeted at each subsequent stage.

Automatic fault detection employs a split ground current so that an imbalance indicates a fault. A faulted component is automatically isolated. Automatic re-routing allows operation with only slightly degraded performance.

An apparatus to treat fluid streams may be summarized as including a first stage non-thermal plasma (NTP) generation cell assembly, the first stage NTP generation cell assembly operable to produce a first NTP field in a first flow path; a second stage NTP generation cell assembly, the second stage NTP generation cell assembly operable to produce a second NTP field in the first flow path positioned relatively downstream from the first NTP field in the first flow path; and a control system coupled to control the first and at least the second NTP generation cell assemblies to respectively produce during a first period: the first NTP field with a first set of NTP field characteristics and the second NTP field with a second set of NTP field characteristics, the second set of NTP field characteristics different than the first set of NTP field characteristics.

The first and the second sets of NTP field characteristics may include frequency, and the control system may be operable to control the first NTP generation cell assembly such that the first NTP field has a first frequency and may be operable to control the second NTP generation cell assembly such that the second NTP field has a second frequency, the second frequency different than the first frequency. The first and the second sets of NTP field characteristics may include at least one of a wave shape or a power level, and the control system may be operable to control the first stage NTP generation cell assembly to have at least one of a first wave shape or a first power level and may be operable to control the second stage NTP generation cell assembly to have at least one of a second wave shape or a second power level, different than the first wave shape or first power level, respectively. The first and the second sets of NTP field characteristics may include frequency, wave shape and power level, and the control system may be configured to control the first NTP generation cell assembly such that the first NTP field characteristics transforms a first compound to a second compound and to control the second NTP generation cell assembly such that the second NTP field characteristics transforms the second compound to a third compound. The first and the second sets of NTP field characteristics may include frequency, wave shape and power level, and the control system may be configured to control the first NTP generation cell assembly such that the first NTP field characteristics destroy a first volatile organic compound (VOC) and to control the second NTP generation cell assembly such that the second NTP field characteristics destroy a second VOC, the second VOC relatively less complicated than the first VOC. The first and the second sets of NTP field characteristics may include frequency, wave shape and power level, and the control system may be configured to control the first NTP generation cell assembly such that the first NTP field characteristics destroy a first number of types of volatile organic compounds (VOCs) and to control the second NTP generation cell assembly such that the second NTP field characteristics destroy a second number of types of VOCs, the second number of types of VOCs being less than the first number of types of VOCs. The apparatus may further include at least one sensor positioned to detect at least one characteristic that is indicative of a first stage ratio between at least one of the first sets of NTP field characteristics and at least one operational characteristic of the first stage NTP generation cell assembly, where the first stage ratio is indicative of an interaction of the first NTP field and a number of contaminants in a fluid stream that passes through the first NTP field. The control system may be configured to identify at least one frequency of the first NTP field at which a power is minimized, and to control the operation the first stage NTP generation cell assembly to oscillate around the identified frequency during operation based at least in part on a feedback indicative of the first stage ratio. The apparatus may further include a third stage NTP generation cell assembly, the third stage NTP generation cell assembly operable to produce a third NTP field in the first flow path positioned relatively downstream from the first and the second NTP fields in the first flow path, and wherein the control system is coupled to control the at least the third NTP generation cell assembly to produce during the first period, the third NTP field with a third set of NTP field characteristics, the third set of NTP field characteristics different than the first and the second sets of NTP field characteristics. The first, the second, and the third sets of NTP field characteristics may include frequency, wave shape and power level, and the control system may be configured to control the first NTP generation cell assembly such that the first NTP field characteristics destroy a first volatile organic compound (VOC), to control the second NTP generation cell assembly such that the second NTP field characteristics destroy a second VOC, and to control the third NTP generation cell assembly such that the third NTP field characteristics destroy a third VOC, the third VOC relatively less complicated than the second VOC, and the second VOC relatively less complicated than the first VOC. The first, the second, and the third sets of NTP field characteristics may include frequency, wave shape and power level, and the control system may be configured to control the first NTP generation cell assembly such that the first NTP field characteristics destroy a first number of types of volatile organic compounds (VOCs), to control the second NTP generation cell assembly such that the second NTP field characteristics destroy a second number of types of VOCs, and to control the third NTP generation cell assembly such that the third field characteristics destroy a third of types of VOCs, the second number of types of VOCs being less than the first number of types of VOCs, and the third number of types of VOCs being less than the second number of types of VOCs. The apparatus may further include a parallel first stage non-thermal plasma (NTP) generation cell assembly, the parallel first stage NTP generation cell assembly operable to produce a parallel first NTP field in a second flow path; and a parallel second stage NTP generation cell assembly, the parallel second stage NTP generation cell assembly operable to produce a parallel second NTP field in the second flow path positioned relatively downstream from the parallel first NTP field in the second flow path, and wherein the control system is coupled to control the parallel first and at least the parallel second NTP generation cell assembly to respectively produce during the first period: the parallel first NTP field with the first set of NTP field characteristics and the parallel second NTP field with the second set of NTP field characteristics. The apparatus may further include a gas inlet positioned relatively upstream of the first stage NTP generation cell assembly; and a gas outlet positioned relatively downstream of the second stage NTP generation cell assembly, wherein the first flow path extends between the gas inlet and the gas outlet. The first stage and the second stage NTP generation cell assemblies may each be planar dielectric barrier discharge (DBD) type NTP generation cell assemblies which respectively include at least one electrically hot electrode and at least two electrically ground electrodes provided in an alternating arrangement, and at least one dielectric barrier spaced between the at least one electrically hot electrode and the at least two electrically ground electrodes to provide at least one gap therebetween, and wherein the at least one gap between the at least one electrically hot electrode and the at least two electrically ground electrodes of each of the first stage and the second stage NTP generation cell assemblies form part of the first flow path. At least one of the at least one electrically hot electrode and the at least two electrically ground electrodes of each of the first stage and the second stage NTP generation cell assemblies may be made of the catalytically active material which is exposed in the first flow path to a fluid stream to be treated during operation of the apparatus and wherein dielectric barriers are one of coated with a catalytically active material or comprised of a catalytically active material.

A method of operating an apparatus to treat fluid streams may be summarized as including operating a first stage non-thermal plasma (NTP) generation cell assembly during a first period to produce a first NTP field in a first flow path with a first set of NTP field characteristics; and operating a second stage NTP generation cell assembly during the first period to produce a second NTP field in the first flow path positioned relatively downstream from the first NTP field in the first flow path with a second set of NTP field characteristics, the second set of NTP field characteristics different than the first set of NTP field characteristics.

The first and the second sets of NTP field characteristics may include frequency, and operating the first NTP generation cell assembly may include operating the first NTP generation cell assembly such that the first NTP field has a first frequency and operating the second NTP generation cell assembly may include operating the second NTP generation cell assembly such that the second NTP field has a second frequency, the second frequency different than the first frequency. The first and the second sets of NTP field characteristics may include at least one of a wave shape or a power level, and operating the first stage NTP generation cell assembly may include operating the first stage NTP generation cell assembly to have at least one of a first wave shape or a first power level and operating the second stage NTP generation cell assembly may include operating the second stage NTP generation cell assembly to have at least one of a second wave shape or a second power level, different than the first wave shape or first power level, respectively. The first and the second sets of NTP field characteristics may include frequency, wave shape and power level, and operating the first NTP generation cell assembly may include operating the first NTP generation cell assembly such that the first set of NTP field characteristics transforms a first compound to a second compound and operating the second NTP generation cell assembly such that the second set of NTP field characteristics transforms the second compound to a third compound. The first and the second sets of NTP field characteristics may include frequency, wave shape and power level, and operating the first NTP generation cell assembly may include operating the first NTP generation cell assembly such that the first NTP field characteristics destroy a first volatile organic compound (VOC) and operating the second NTP generation cell assembly may include operating the second NTP generation cell assembly such that the second NTP field characteristics destroy a second VOC, the second VOC relatively less complicated than the first VOC. The first and the second sets of NTP field characteristics may include frequency, wave shape and power level, and operating the first NTP generation cell assembly may include operating the first NTP generation cell assembly such that the first NTP field characteristics destroy a first number of types of volatile organic compounds (VOCs) and operating the second NTP generation cell assembly may include operating the second NTP generation cell assembly such that the second NTP field characteristics destroy a second number of types of VOCs, the second number of types of VOCs being less than the first number of types of VOCs. The method may further include detecting a characteristic that is indicative of a first stage ratio of at least one characteristic of the first set of NTP field characteristics and at least one operational characteristic of the first stage NTP generation cell assembly, where the first stage ratio is indicative of an interaction of the first NTP field and a number of contaminants in a fluid stream that passes through the first NTP field; identifying at least one frequency of the first NTP field at which a power is minimized; and controlling the operation the first stage NTP generation cell assembly to oscillate around the identified frequency during operation based at least in part on a feedback signal indicative of the first stage ratio. The method may further include operating a third stage NTP generation cell assembly during the first period to produce a third NTP field in the first flow path positioned relatively downstream from the first and the second NTP fields in the first flow path with a third set of NTP field characteristics, the third set of NTP field characteristics different than the first and the second sets of NTP field characteristics. The first, the second, and the third sets of NTP field characteristics may include frequency, wave shape and power level, and operating the first NTP generation cell assembly may include operating the first NTP generation cell assembly such that the first NTP field characteristics destroy a first volatile organic compound (VOC), operating the second NTP generation cell assembly may include operating the second NTP generation cell assembly such that the second NTP field characteristics destroy a second VOC, and operating the third NTP generation cell assembly may include operating the third NTP generation cell assembly such that the third NTP field characteristics destroy a third VOC, the third VOC relatively less complicated than the second VOC, and the second VOC relatively less complicated than the first VOC. The first, the second, and the third sets of NTP field characteristics may include frequency, wave shape and power level, and operating the first NTP generation cell assembly may include operating the first NTP generation cell assembly such that the first NTP field characteristics destroy a first number of types of volatile organic compounds (VOCs), operating the second NTP generation cell assembly may include operating the second NTP generation cell assembly such that the second NTP field characteristics destroy a second number of types of VOCs, and operating the third NTP generation cell assembly may include operating the third NTP generation cell assembly such that the third field characteristics destroy a third of types of VOCs, the second number of types of VOCs being less than the first number of types of VOCs, and the third number of types of VOCs being less than the second number of types of VOCs. The method may further include operating a parallel first stage non-thermal plasma (NTP) generation cell assembly during the first period to produce a parallel first NTP field in a second flow path with the first set of NTP field characteristics; and operating a parallel second stage NTP generation cell assembly during the first period to produce a second NTP field in the second flow path positioned relatively downstream from the parallel first NTP field in the second flow path with the second set of NTP field characteristics.

A method of operating an apparatus to treat fluid streams may be summarized as including operating a first stage non-thermal plasma (NTP) generation cell assembly during a first period to produce a first NTP field in a first flow path with a first set of NTP field characteristics; detecting a characteristic that is indicative of a first stage ratio of at least one characteristic of the first set of NTP field characteristics and at least one operational characteristic of the first stage NTP generation cell assembly, where the first stage ratio is indicative of an interaction of the first NTP field and a number of compounds in a fluid stream that passes through the first NTP field; identifying at least one frequency of the first NTP field at which the first stage ratio is optimized; and controlling the operation of the first stage NTP generation cell assembly to oscillate around the identified frequency during at least one subsequent period based at least in part on a feedback indicative of the first stage ratio.

Operating a first stage NTP generation cell assembly during a first period may include operating the first stage NTP generation cell assembly to step through a first range of frequencies for the first NTP field, and controlling the operation of the first stage NTP generation cell assembly to oscillate around the identified frequency during at least one subsequent period includes controlling the operation of the first stage NTP generation cell assembly to oscillate the first NTP field around the identified frequency in a second range, smaller than the first range. Identifying at least one frequency of the first NTP field at which the first stage ratio is optimized may include identifying at least one frequency of the first NTP field at which a power of the first NTP field increases without a corresponding change in the operational characteristics of the first stage NTP generation cell assembly. Identifying at least one frequency of the first NTP field at which the first stage ratio is optimized may include identifying at least one frequency of the first NTP field at which a power of the first NTP field is maintained in response to a decrease of a voltage applied by the first stage NTP generation cell assembly. The interaction that the first stage ratio is indicative of may include an interaction of the first NTP field and a number of volatile organic compound contaminants in a fluid stream that passes through the first NTP field.

### BRIEF DESCRIPTION OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a schematic diagram of a non-thermal plasma generation portion of a fluid flow treatment system and a control portion thereof, which employs a number of non-thermal plasma generation cell assemblies to treat fluid flows, according to one illustrated embodiment.

FIG. 2 is a functional block diagram of a portion of a fluid flow treatment system illustrating a power supply used to provide high voltage to electrodes of a non-thermal plasma generation cell assembly, according to one illustrated embodiment.

FIG. 3 is an isometric view of a portion of a fluid flow treatment system having three parallel flow paths, each parallel path having two electrode assemblies arranged in series to produce successive non-thermal plasma fields, according to one illustrated embodiment.

FIG. 4A an isometric view of a portion of one flow path of a fluid flow treatment system showing a housing, two electrode assemblies and one high voltage transformer, according to one illustrated embodiment.

FIG. 4B is an enlarged view of a portion of the a non-thermal plasma generation cell assembly of FIG. 4A, showing two electrode assemblies.

FIG. 5 is a partially exploded isometric view of an electrode assembly, according to one illustrated embodiment.

FIG. 6A is a plan view of an electrode suitable for use in an electrode assembly, according to one illustrated embodiment.

FIG. 6B is an enlarged view of a portion of an electrically conductive element of the electrode of FIG. 6A.

FIG. 6C is an enlarged view of an electrical connector or terminal of the electrode of FIG. 6A.

FIG. 7 is a plan view of a hot electrode overlying a ground electrode to form as least part of an electrode assembly, according to one illustrated embodiment.

FIG. 8 is a schematic view of a portion of a fluid flow treatment system illustrating a portion of a fault detection subsystem, according to one illustrated embodiment.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with fluid flow systems such as conduits, dampers, vents, fans, compressors, or blowers have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its broadest sense, that is as meaning “and/or” unless the content clearly dictates otherwise.

The headings and Abstract of the Disclosure provided herein are for convenience only and do not interpret the scope or meaning of the embodiments.

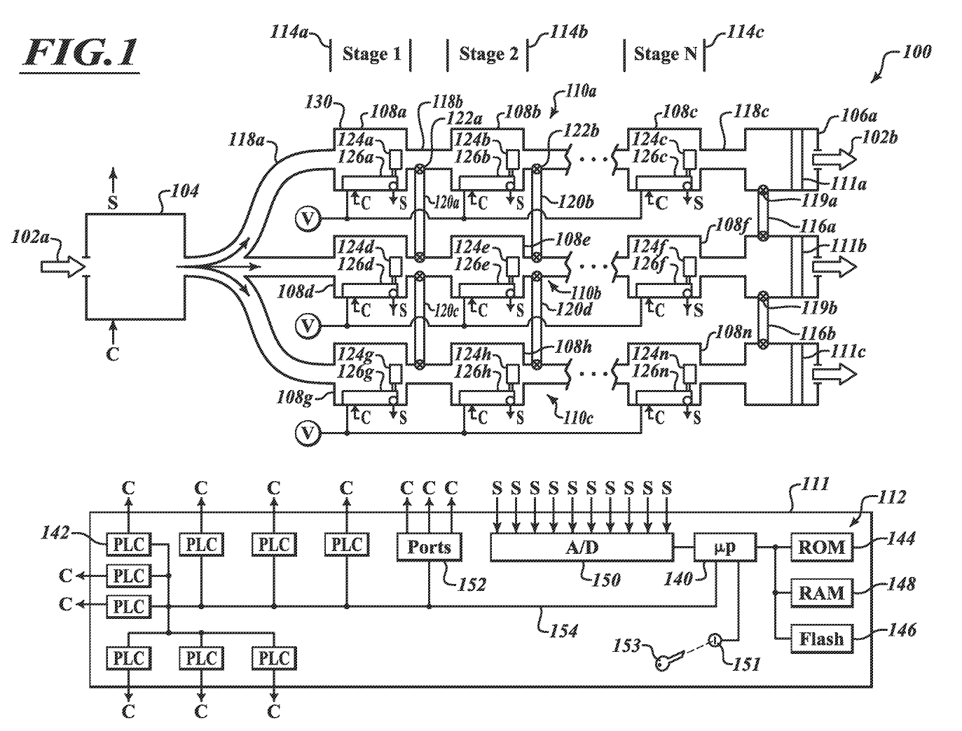

FIG. 1 shows a fluid flow treatment system 100, according to one illustrated embodiment. As described herein, the fluid flow treatment system 100 may be used to treat fluid flows (arrows 102a, 102b, collectively 102) for example streams of gases (e.g., air) resulting from sources (not shown), for instance resulting from various manufacturing processes or other processes. Such may advantageously transform contaminates or pollutants to safe or safer forms, or even eliminate contaminates or pollutants.

The fluid flow treatment system 100 includes a flow input 104, a number of flow outputs 106a-106c (three illustrated, collectively 106), a plurality of non-thermal plasma (NTP) generation cell assemblies 108a-108n (nine illustrated, collectively 108) located in one or more flow paths 110a-110c (three illustrated, collectively 110) between the flow input 104 and a respective flow output 106, and a control subsystem 112 communicatively coupled to control various elements of the fluid flow treatment system 100. The NTP generation cell assemblies 108 are arranged, as illustrated, in a number of stages 114a-114c (three illustrated, collectively 114) located relatively along the flow paths 110 between the flow input 104 and the respective flow output 106, each stage 114 advantageously controlled to target a respective pollutant or pollutants. The NTP generation cell assemblies 108 may also optionally be arranged, as illustrated, in a number of parallel flow paths 110a-110c. The use of two or more parallel flow paths 110 may allow treatment of relatively large volumes of fluid flow.

As best illustrated in FIG. 1, the flow input 104 is fluidly communicatively coupled to a source (not shown) of a fluid flow 102a, for example one or more pieces of industrial machinery. The flow input 104 may be a simple duct, connector, or hood positioned to collect a fluid flow to be treated. The flow input 104 may take the form of a manifold, for example distributing portions of the fluid flow to respective parallel flow paths 110. The flow input 104 may, for example, include one or more actuators (not shown in FIG. 1). For instance, the flow input 104 may include a fan, compressor, or blower (not shown in FIG. 1). Such may be employed to cause movement of the fluid flow along the fluid or flow path(s) 110. Also for instance, the flow input 104 may include a motor or solenoid (not shown in FIG. 1) coupled to control a damper and/or a vent (not shown in FIG. 1). Such may be used to selectively open and/or close certain of the parallel flow paths 110.

The flow output 106 may vent treated fluid flow 102b to the ambient environment or to other environments. While illustrated as separate flow outputs 106a-106c, the fluid flow treatment system 100 may combine separate parallel flow paths 110 into a single flow output 106. The flow output 106 typically includes one or more catalysts, for instance an ozone destruct catalyst 111a-111c (three shown, collectively 111).

The flow output 106 may, for example, include one or more actuators (not shown in FIG. 1). For instance, the flow output 106 may include a fan, compressor, or blower (not shown in FIG. 1). Such may be employed to cause movement of the fluid flow along the fluid or flow path(s) 110, out to the ambient environment, and/or feedback flow conduit 116. Also for instance, the flow output 106 may include a motor or solenoid (not shown in FIG. 1) coupled to control a damper and/or a vent (not shown in FIG. 1). Such may be used to selectively vent to the ambient environment.

A number of cross output flow paths 116a, 116b (two shown, collectively 116) may provide selectively provide fluidly communicative paths between the flow outputs 106a-106c, or portions thereof. The cross output flow paths 116 may include a vent, damper or valve 119a, 119b (four shown, two called out, collectively 119), selectively operable via an actuator (e.g., electric motor, solenoid) to open or close the respective cross output flow paths 116. The cross output flow paths 116 may allow selective bypassing of one or more of the flow output 106 and associated catalyst 111, for example where the catalyst 111 has become poisoned, or is being replaced or reconditioned. Such may advantageously allow re-routing of flow as needed to address contingencies or even to accommodate different applications or installations.

In some implementations, a return conduit (not shown) provides a feedback flow path. Such may allow fluid flows to be retreated. Flow along the return conduit may be selectively controlled via one or more actuators (not shown in FIG. 1), for example a motor or solenoid (not shown in FIG. 1) coupled to control a damper and/or a vent (not shown in FIG. 1).

The parallel flow paths 110 may be defined by two or more stages of NTP generation cell assemblies 108 and any conduits 118a-118c (only three called out, collectively 118) coupled therebetween or between the NTP generation cell assemblies 108 and the flow input 104 or flow output 106. The conduits 118 may take any form suitable to direct fluid flow along a desired flow path.

One or more transverse intermediate flow paths 120a-120d (four illustrated, collectively 120) may provide selectively provide fluidly communicative paths between the parallel flow paths 110, or portions thereof. The transverse intermediate flow paths 120 may include a vent, damper or valve 122a, 122b (two illustrated, collectively 122), selectively operable via an actuator (e.g., electric motor, solenoid) to open or close the respective transverse intermediate flow paths 120. The transverse intermediate flow paths 120 may allow selective bypassing of one or more of the NTP generation cell assemblies 108, for example where one of the NTP generation cell assemblies 108 is malfunctioning or being repaired or reconditioned.

The NTP generation cell assemblies 108 generally include one or more sets of electrodes or electrode assemblies 124a-124n (nine illustrated in FIG. 1, collectively 124) and a respective power supply 126a-126n (nine illustrated in FIG. 1, collectively 126). The power supplies 126 are electrically coupled to provide high voltage electrical power to electrodes of the respective electrode assemblies 124 to produce a non-thermal plasma between or proximate the electrodes having a desired frequency. In a non-thermal plasma, the electron flow is primarily due to voltage, rather than heat. As described further herein, the frequency of the non-thermal plasma may be selected to match or be a harmonic of a resonant frequency associated with a particular pollutant (e.g., compound, volatile organic compound of VOC) that is targeted to be reduced or destroyed at the particular stage 114.

The NTP generation cell assemblies 108 for any given parallel flow path 110 may be housed in respective housings 130 (only one called out), as illustrated in FIG. 1. Alternatively, the two or more NTP generation cell assemblies 108 for any given parallel flow path 110 may be co-housed in a common housing. The housing(s) is or are designed to cause a fluid flow stream to pass over or by the electrode assemblies 124 through the non-thermal plasma. The housings may comprise a simple sheet metal structure.

As described in more detail herein, the respective power supplies 126 may be controlled to supply electrical power having certain selected or defined electrical characteristics to respective electrode assemblies 124. The power supply 126 may, for example, control a voltage and a frequency of the electrical power supplied to the respective electrode assembly 124. The power supply 126 may additionally control an amperage and/or phase of the electrical power supplied to the respective electrode assembly 124. Control of frequency may advantageously be employed to target different pollutants (e.g., different organic compounds such as volatile organic compounds or VOCs). For example, each successive power supply 126 in a respective parallel flow path 110 may supply electrical current at a respective frequency (e.g., 50 Hz-50 KHz) to the respective electrode assembly 124, to match a resonant frequency or harmonic of a targeted compound. Controlling the electrical characteristics may also allow a desired power density (e.g., Watts/Area) to be achieved at or proximate the electrode assemblies 124. Sufficiently high power densities may be useful in decomposing certain compounds and/or in incinerating or otherwise destroying certain PMₓₓ particulate. For instance, sufficiently high power densities may be used to small (e.g., 2.5 microns) particulate, for example smoke. Notably, such is expected to be effective at treating with PMₓₓ particulate that is organic and that can be incinerated.

The power supply 126 is electrically coupled to receive electrical power from a power source V. The power source V may be an electrical mains, circuit box or other source of electrical power such as an electrical grid. The power source V will typically supply alternating current (AC) power, which may be three phase electrical power at 60 Hz. Alternatively, in some implementations a single phase or two phase AC power service may be employed.

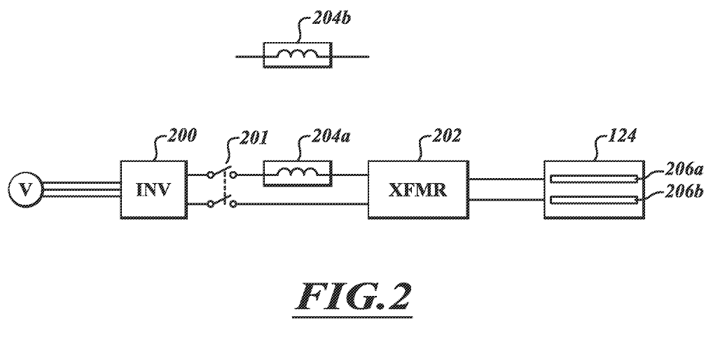

The power supply 126 may step up voltage, step down voltage, rectify, invert, condition or otherwise convert the received electrical power before supplying the converted electrical power to the electrode assemblies 124 of the respective NTP generation cell assembly 108. As illustrated in FIG. 2, each power supply 126 may include one or more inverters 200 (one illustrated), contactor 201 high voltage/high frequency transformers 202 (one illustrated), and optionally tuning inductors 204a, 204b (two shown, collectively 204). The inverter 200 is electrically coupled to receive AC power from a power source V. The inverter 200 is controlled to selectively adjust a frequency of the electrically current. The inverter 200 may for example be controlled via one or more PLCs. The contactor(s) 201 are controlled to selectively couple the AC power from the inverter 200 to the high voltage/high frequency transformer 202. The contactor(s) 201 may be controlled by the control subsystem.

The high voltage/high frequency transformer 202 is electrically coupled to step up a voltage of the electrical current from the inverter 200 to levels sufficiently high to generate a non-thermal plasma when applied across the electrodes 206a, 206b (two shown, collectively 206) of the electrode assembly 124. For example, the high voltage/high frequency transformer 202 may step up voltage to approximately 8 Kilo-Volts (KV).

One or more tuning inductors 204 may be used to tune the power supply 126 based on the particular characteristics of the electrode assembly 124 and electrodes 206 thereof. The tuning inductor 204 may be used to balance the capacitance of the electrodes with the inductance of the high voltage transformer 202 and other inductances in the circuit to achieve a desired frequency. Such advantageously allows use of a single high voltage transformer design with a large variety of different electrode assembly designs and materials. Commercial installations will typically have a single tuning inductor 204 for each transformer. However, some embodiments may include two or more tuning inductors 204. These tuning inductors may be 204 switchable into, and out of, the circuit. This may allow identification of resonant frequencies for new compounds or other pollutants. For example, tuning inductors 204 may selectively tried, stepping through various center frequencies to find a resonant state of a compound.

In some implementations, the power supply 126 may optionally additionally include one or more boost converter circuits, buck converter circuits, buck-boost converter circuits, flyback converter circuits, rectifier circuits, alternator circuits, conditioner circuits. One or more of these circuits may be passive (e.g., diode rectifier bridge). One or more of these circuits may be active (e.g., metal oxide semiconductor field effect transistor MOSFET or insulated gate bipolar transistor or IGBT full or half bridge circuit).

Returning to FIG. 1, active electrical or electronic devices, for instance the inverter 200, may be driven responsive to one or more control signals C. As discussed in more detail below, control signals may be generated by the control subsystem 112.

The NTP generation cell assemblies 108 may include one or more sensors S (one illustrated per NTP generation cell assembly). The sensors S may take a variety of forms, and may sense a large variety of characteristics, conditions or states. For example, one or more sensors may sense electrical characteristics. For instance, one or more sensors S may be associated with a respective one of the power supplies 126 to sense electrical characteristics thereof. The sensors may include one or more a current sensors, voltage sensors, resistance sensor, or impedance sensors. Also for example, one or more sensors S may sense characteristics of the fluid flow. The sensor S may include one or more volumetric flow sensors, pressure sensors or speed sensors (e.g., anemometer). In this respect, it is noted that it may be important to maintain a pressure gradient across the NTP generation cell assembly 108 given the amount of energy in the non-thermal plasma field. Thus, one or more volumetric flow sensors, pressure sensors or speed sensors may be monitored to assure that there is sufficiently flow for the power. As a further example, one or more sensor S may sense an operational condition of a mechanical or electromechanical element, for instance a position of a vent or damper. The sensors S may include one or more position sensors, contact switches, optical sensors, encoders or other types of sensors.

The control subsystem 112 is preferably housed in separate controller cabinetry 111 from the power electronics, and particular from high voltage transformers to prevent interference with the circuitry of the control subsystem 112. The control subsystem 112 may take large variety of forms, including various hardware, firmware and/or software components. The control subsystem 112 may include one or more controllers such as a microprocessor 140 or one or more programmable logic units (PLC) 142 (nine illustrated, only one called out). The control subsystem 112 may include one or more nontransitory computer- or processor-readable mediums. For example, the control subsystem 112 may include nonvolatile memory such as a read only memory (ROM) 144 or FLASH memory 146. Also, for example, the control subsystem 112 may include volatile memory such as a dynamic random access memory (RAM) 148.

The control subsystem 112 may optionally include one or more analog-to-digital converts 150, for instance where sensors S provide sensed information in an analog form. The control subsystem 112 may include one or more ports 152 (only one illustrated) to provide communications therefrom. For example, one or more serial or parallel ports 152 may provide communications with various components of the rest of the fluid flow treatment system 100 or with devices or components external thereto and/or separate therefrom.

The various components may be coupled via one or more buses 154 or other architectures. While only one bus 154 is illustrated, typically there will be separate buses for different functions, such as a power bus, communications bus, instruction bus, address bus, data bus, etc.

The microprocessor 140 may control various elements of the remainder of fluid flow treatment system 100. For example, the microprocessor 140 may provide control signals C to control various actuators, for instance based signals from sensor S representative of sensed information, conditions, or states. The microprocessor 140 may detect or monitor for faults or other abnormalities. For example, the microprocessor 140 may monitor one or more sensors to ensure there is sufficient flow across the NTP generation cell assemblies 108 or to ensure that no ground fault, shorting or arcing has occurred. The microprocessor 140 may take appropriate action on occurrence or detection of a fault or abnormality. For instance, the microprocessor 140 may provide control signals C to one or more actuators to open or close dampers or valves to route fluid flow past a faulty component (e.g., NTP generation cell assembly 108), and/or adjust a speed of a fan, compressor, or blower. Also for instance, the microprocessor 140 may provide control signals to shut off power to one or more components (e.g., high voltage transformers 202, electrode assemblies 124) associated with a particular one of the NTP generation cell assemblies 108 in response to detection of insufficient flow across the particular NTP generation cell assemblies 108 and/or to cause a notification or alert to be provided or sent. The microprocessor 140 may provide control signals C to shut down all or part of the fluid flow treatment system 100 on occurrence of an electrical problem, such as a ground fault. For instance, the microprocessor 140 may provide control signals C to stop operation of one or more components, open contactors to shut off power to one or more components (e.g., high voltage/high frequency transformers 202, electrode assemblies 124) and/or to cause a notification or alert to be provided or sent. The microprocessor 140 may optionally control the PLCs, for example providing desired settings for frequencies and/or voltages to the PLCs 142. The microprocessor 140 may take any of a large variety of forms, including various complex instruction set or reduced instruction set microprocessor sold by INTEL, Motorola, Advanced Micro Devices, etc.

The PLCs 142 are communicatively coupled to control operation of respective ones of the inverters 200 (FIG. 2). The PLCs 142 can provide control signals C to cause respective ones of the inverters 200 to adjust a frequency of the output current of the inverter 200. The PLCs 142 may take any of a large variety of forms, including commercially available PLCs such as those sold by Siemens.

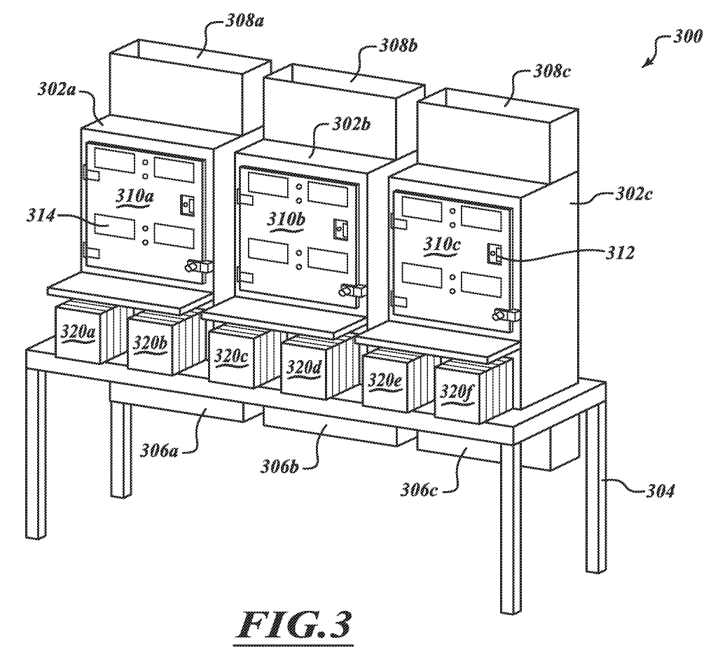

The control subsystem 112 may include a key activated switch 151 which requires the presence of a key 153 to allow power to be supplied to the high voltage/high frequency transformers 202. The key 153 may be a mechanical key or may be an electronic key. The key activated switch 151 and key 153 may be used to ensure safe operation, as discussed below in reference to FIG. 3.

FIG. 3 shows a portion of a fluid flow treatment system 300, according to one illustrated embodiment. FIG. 3 omits illustration of the control subsystem previously illustrated and described, as well as an associated control cabinet which may house the control subsystem.

The fluid flow treatment system 300 includes three distinct housings 302a-302c (collectively 302). The housings 302 may be supported by a frame or other support assembly 304.

Each housing 302 has an inlet 306a-306c (collectively 306), and outlet 308a-308c (collectively 308), the housing 302 forming an enclosed interior space or volume (not visible in FIG. 3) extending between the inlet 306 and the outlet 308. In some installations the designation of the inlet and outlet may be reversed from that illustrated. Each housing 302 may have a door 310a-310c (collectively 310) to selectively provide access to the interior space or volume, for example to access electrode assemblies (not visible in FIG. 3) located therein. Each door may include a lock 312 (three shown, only one called out in FIG. 3). The lock 312 requires a key 153 (FIG. 1) to open. Advantageously, the same key 153 may be required to power the fluid flow treatment system 300, as noted above. This may be combined with a cutoff switch that will not allow the fluid flow treatment system 300 to operate with the door 310 open, ensuring safe operation. In particular, the lock 312 may be captive in a key switch such that when the key is removed to open a door (not shown) of a controller cabinet 111 (FIG. 1) that houses control subsystem 112 (FIG. 1), the control subsystem 112 disables power to the high voltage/high frequency transformer(s) 320. The control subsystem 112 may also, responsive to such, switches in ground contacts (not shown), rendering the controller cabinet 111 electrically safe prior to anyone being able to open the door and gain access to high voltage wiring and terminals.

The housings 302 may each include a number of windows 314 (twelve shown, only one called out in FIG. 3). The windows 314 may be aligned with respective plasma fields generated by the electrode assemblies, allowing visual inspection of the plasma fields during operation.

In the embodiment illustrated in FIG. 3, each housing 302 has two high voltage/high frequency transformers 320a-320f (collectively 320). Each of the high voltage/high frequency transformers 320 is electrically coupled to provide electrical power to a respective stage of electrode assemblies. Thus, in this illustrated embodiment each housing 302 includes two stages of electrode assemblies (not visible in FIG. 3), each stage supplied by a respective one of the high voltage/high frequency transformers 320. Each stage may include one, two or more sets of electrodes (not visible in FIG. 3).

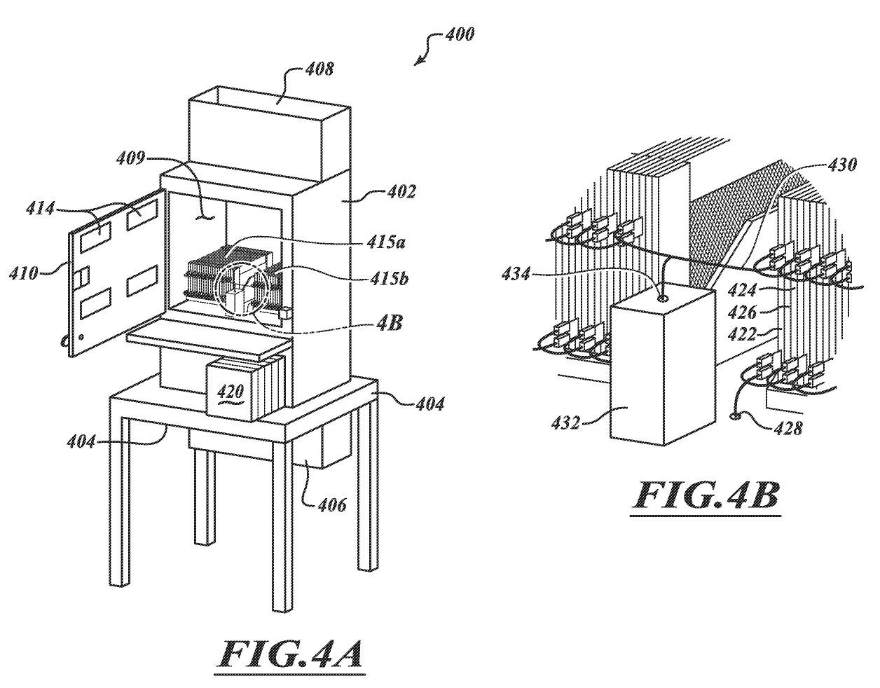

FIGS. 4A and 4B show a portion of a fluid flow treatment system 400, according to one illustrated embodiment. FIG. 4A omits illustration of the control subsystem previously illustrated and described, as well as an associated control cabinet which may house the control subsystem.

The fluid flow treatment system 400 includes a single distinct housings 402. The housing 402 may be supported by a frame or other support assembly 404.

The housing 402 has an inlet 406, and outlet 408, the housing 402 forming an enclosed interior space or volume 409 extending between the inlet 406 and the outlet 408. In some installations the designation of the inlet and outlet may be reversed from that illustrated. The housing 402 may have a door 410 to selectively provide access to the interior space or volume 409, for example to access electrode assemblies 422 (only) located therein. The housing 402 may include a number of windows 414 (two shown), aligned with respective plasma fields generated by the electrode assemblies 415a, 415b (two shown, collectively 415), allowing visual inspection of the plasma fields during operation.

In the embodiment illustrated in FIGS. 4A and 4B, the housing 402 has a number of high voltage/high frequency transformers 420 (one shown in FIG. 4A). Each of the high voltage/high frequency transformers 420 is electrically coupled to provide electrical power to a respective stage of electrode assemblies 415. The electrode assemblies 415 include one or more pairs of ground electrodes 422 (only one called out in FIG. 4B) and hot electrodes 424 (only one called out in FIG. 4B), as well as electrically insulative dielectric barrier plates 426 (only one called out in FIG. 4B). The high voltage/high frequency transformer 420 is electrically coupled to a ground electrode 422 via a ground wire or cable 428 and to a hot electrode 424 via a hot wire or cable 430. Other ground electrodes of an electrode assembly 415 are coupled electrically coupled to one another. The other hot electrodes are coupled electrically to one another. The hot wire or cable 430 may run through an insulative block 432 having a passage 434 therethrough. The insulative block 432 helps prevent shorting or arcing of high voltage current to any metal. The insulative block 432 may, for example, be formed of ultra-high molecular-weight (UHMW) polyethylene.

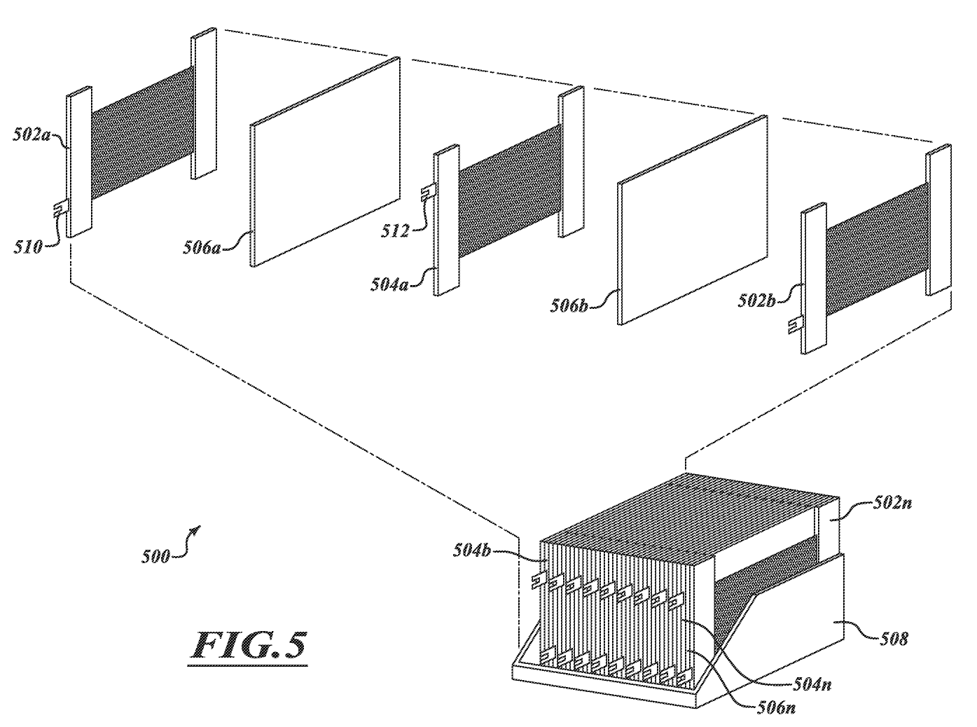

FIG. 5 shows an electrode assembly 500, according to one illustrated embodiment.

Similar to that illustrated in FIG. 4, the electrode assembly 500 includes a number of ground electrodes 502a, 502b-502n (three called out, collectively 502), a number of hot electrodes 504a, 504b-504n (three called out, collectively 504) and a number of dielectric barrier plates 506a, 506b-506n (three called out, collectively 506). As explained below, the ground and hot electrodes 502, 504 may be identical in construction, only the arrangement and electrically coupling causing them to function as ground and hot electrodes, respectively.

The electrode assembly 500 may include a support structure such as a frame 508, to support the ground electrodes 502, hot electrodes 504 and dielectric barrier plates 506.

The electrode assembly 500 may include, starting from an outside thereof, a ground electrode 502, a dielectric barrier plate, and a hot electrode 504, in succession, arranged successively along a dimension of the electrode assembly 500. The dielectric barrier plates 506 are positioned between successive ones (i.e., nearest neighbor) of the ground and hot electrodes 502, 504. Thus, each ground electrode 502 is separated from a nearest hot electrode 504 by a dielectric barrier plate 506. The dielectric barrier plates 506 prevent shorting or arcing between ground and hot electrodes 502, 504. The dielectric barrier plates 506 may optionally include a catalyst or be partially or wholly coated by a catalyst. Suitable catalysts may include TiO₂, formed as an oxide on the surface of the dielectric barrier plate 506, and which is particularly good at forming oxygen radicals.

As illustrated in FIG. 5, each electrode 502, 504 has an electrical connector or terminal 510, 512, respectively (one called out for each of the ground and hot electrodes 502, 504). The electrical connector or terminal 510, 512 allow electrical connections or coupling to the high voltage transformer 420 (FIG. 4). Notably, the electrical connector or terminal 510 of the ground electrodes 502 are spaced apart from the electrical connector or terminal 512 of the hot electrodes 504. For example, the electrical connector or terminal 510 of the ground electrodes 502 may be positioned relatively toward or proximate a bottom of the electrode assembly 500, while the electrical connector or terminal 512 of the hot electrodes 504 are positioned relative toward or proximate a top of the electrode assembly 500. Spacing the electrical connector or terminal 510 of the ground electrodes relatively apart from the electrical connector or terminal 512 of the hot electrodes 504 may reduce the possibility of shorting or arcing. Spacing the electrical connector or terminal 512 of the hot electrodes 504 toward the top helps electrically isolate the electrical connector or terminal 512 from the frame 508 and/or housing 130.

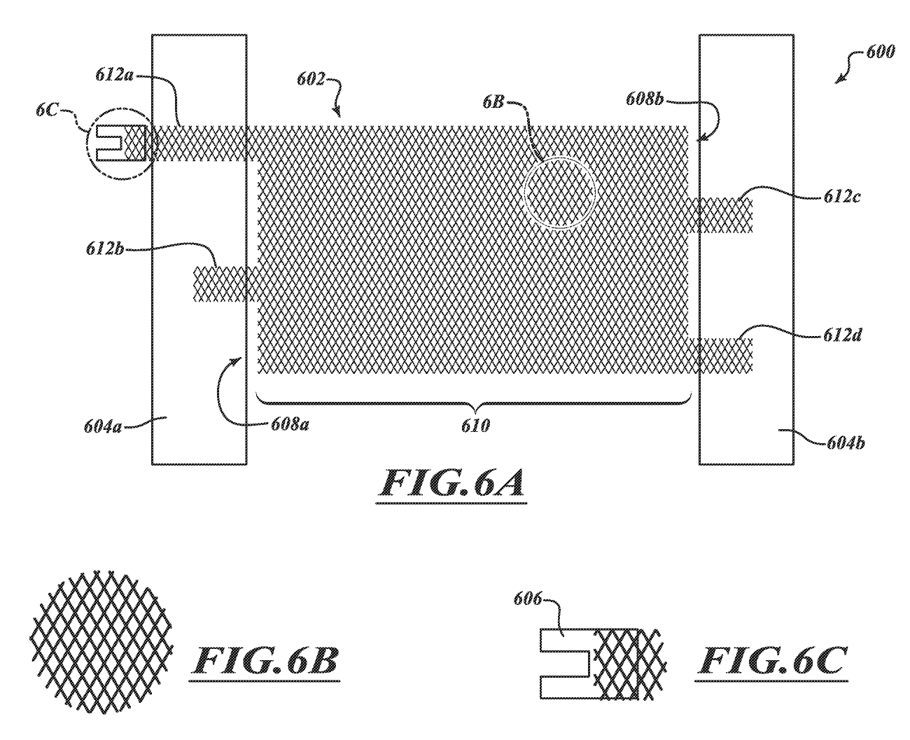

FIG. 6A-6C show an electrode 600 suitable for use in an electrode assembly 502, 504 (FIG. 5), according to one illustrated embodiment.

The electrode 600 includes an electrically conductive substrate 602, a pair of electrically insulative supports 604a, 604b, and an electrical connector or terminal 606. The electrically insulative supports 604a, 604b are positioned at opposed ends 608a, 608b of the electrically conductive substrate 602. The electrically insulative supports 604a, 604b support the electrically conductive substrate 602 while electrically insulating the electrically conductive substrate 602 from other structures, for instance frame 508 (FIG. 5).

The electrically conductive substrate 602 may take the form of a wire mesh or screen, formed of an electrically conductive material such as metal. Applicant has recognized that the use of mesh or screen or similar open structure may be advantageous over solid plates. Applicant believes that improved plasma formation (e.g., 4 times) may be realized via the use of a mesh or screen over a solid plate. The mesh has many edges, at which plasma displays a preference for forming. Without being bound by theory, applicant believes this may be due to micro-turbulence resulting from the structure and/or the extensive amount of edge, on which plasma forms. The electrically conductive substrate 602 may be substantially planar, having a main body portion 610, with legs 612a-612d (collectively 612) extending therefrom at each of the opposed ends 608a, 608b. There may be two legs 612 extending from each end 608a, 608b, which allows the electrically conductive substrate 602 to be secured in the respective insulative supports 604a, 604b. One of the legs 612a is longer than the other legs 612b-612d, and is sufficiently long to extend through the respective insulative support 604a. The electrical connector or terminal 606 is physically and electrically coupled at a terminus of this leg 612a. For example, the electrical connector or terminal 606 may be soldered or welded to the terminus of the leg 612a. The electrical connector or terminal 606 may take any of a large variety of forms that permit physical and electrical connections to high voltage power sources, for instance to a high voltage power transformer 420 (FIG. 4). The electrical connector or terminal 606 may, for instance, take the form of a spade connector, as illustrated in FIGS. 6A and 6C.

The size or surface area of the electrodes 600 should be selected based on a size or dimensions of the dielectric barrier plates 506. The electrodes 600 should not extend beyond the edge(s) or perimeter of the dielectric barrier plates 506 when arranged, aligned or placed in registration in a stack or other arrangement. The size or dimensions of the dielectric barrier plates 506 may vary on the particular application as well as on the commercial offerings of various sources of dielectric barrier plates 506.

The electrically conductive portion of each of electrode 600 can be made of a metal catalyst, for instance titanium or copper. Alternatively, the electrically conductive portion may be formed of other electrically conductive materials (e.g., other metals), coated with a catalyst. The electrically conductive portion may, for example, take the form of a titanium base with or without a catalyst coated on the titanium. The electrically conductive portion may, for example, take the form of metal other than titanium and coated with a catalyst. The metal should be compatible with the particular catalyst employed. Any metal catalyst that can be vaporized on, electroplated on, or otherwise deposited on, or formed into the electrically conductive portion may be employed. Suitable metal catalysts may include Fe, Co, Ni, Cu, Ru, Rh, Pd, Ag, Ir, Pt and Au, as well as combinations or alloys thereof, for instance an alloy of Pt and Rh. Non-metallic catalysts may also be employed. Suitable non-metallic catalyst may include chromium oxide or aluminum oxide.

The electrically conductive portion of each electrode 600 may, for example, be approximately 0.03 inches thick. An open area of the electrically conductive portion may vary, for example from approximately 50% to approximate 75% of a total area of the exposed electrically conductive portion. The electrically conductive portion maybe selected from various commercially available meshes, which may reduce cost.

The total number of ground and/or hot electrodes 502, 504, and hence the total number of dielectric barrier plates 506, may be dependent on the particular application or installation and/or on the surface area of the electrodes 502, 504. For some applications approximately ten of each type of electrode 502, 504 may be suitable.

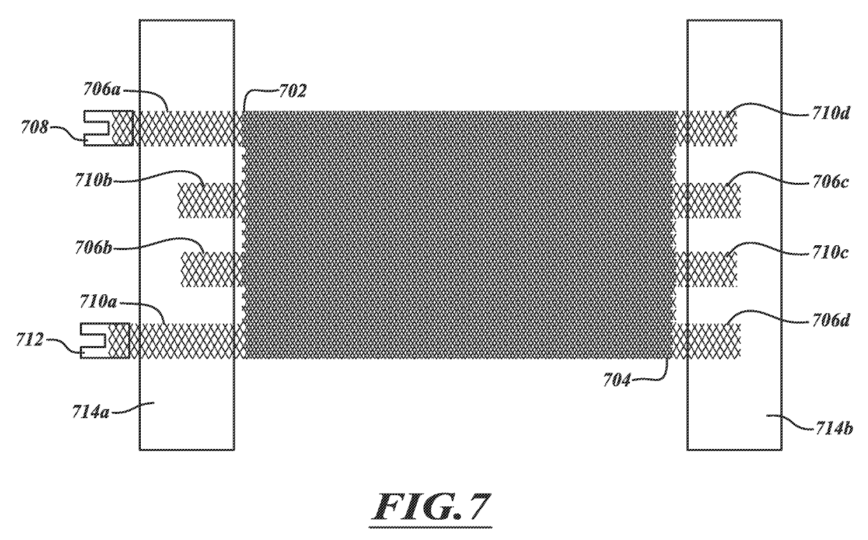

The illustrated electrode 600 may advantageously be used as either a ground electrode 502 (FIG. 5) or hot electrode 504 (FIG. 5), dependent on the orientation of the electrode 600 in the electrode assembly 500 (FIG. 5). Such is best illustrated in FIG. 7.

FIG. 7 shows a pair of two identical electrodes, first electrode 702 and second electrode 704 arranged with respect to one another to function as a hot electrode (e.g., electrode 702) and a ground electrode (e.g., electrode 704), according to one illustrated embodiment.

The first electrode 702 has a long leg 706a, three short legs 706b-706d, and electrical connector or terminal 708. The second electrode 704 has a long leg 710a, three short legs 710b-710d, and electrical connector or terminal 712. Electrically insulative supports 714a, 714b of the hot electrode are visible in FIG. 7.

The first and second electrodes 702, 704, respectively, may advantageously be identical in structure, shape and constitution. Notably, the second electrode 704 is oriented in the electrode assembly in an opposite orientation (flipped about either a longitudinal or a lateral axis) from the orientation of the first electrode 702. Thus, while the long leg 706 and electrical connector or terminal 708 of the first electrode 702 is positioned relatively toward the top of FIG. 7, the long leg 710 and electrical connector or terminal 712 of the second electrode 704 is positioned relatively toward the bottom of FIG. 7. Thus, an identical electrode structure may advantageously be used to implement both the ground and the hot electrodes 502, 504 (FIG. 5), respectively, in an electrode assembly 500 (FIG. 5) simply by changing the orientation of the electrode structure in the electrode assembly 500 and making the appropriate electrical connections.

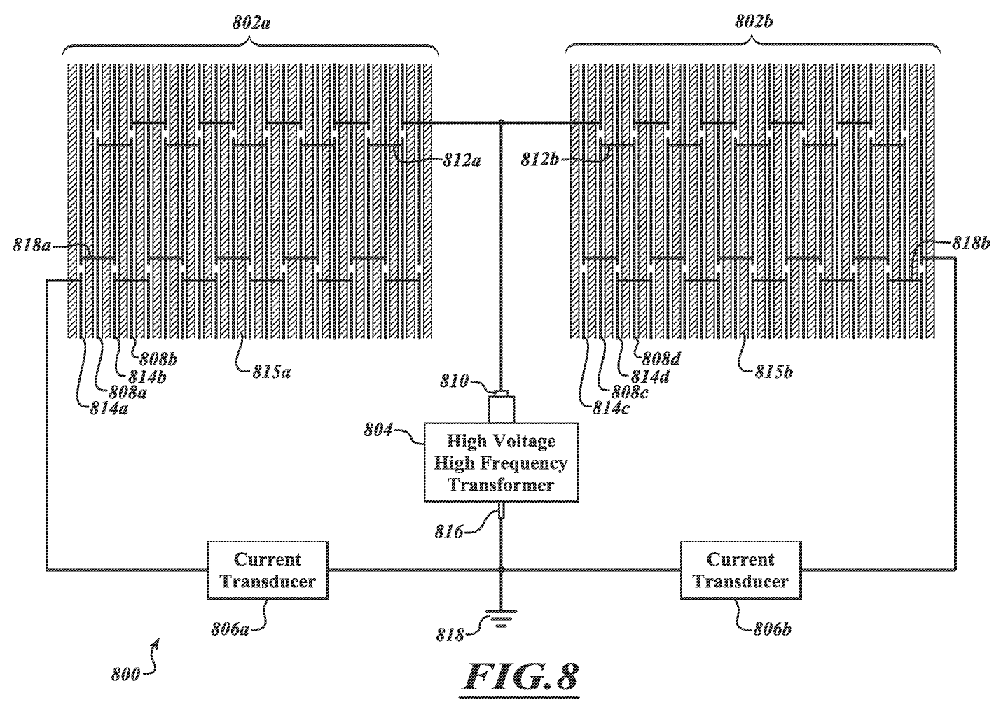

FIG. 8 shows a portion of a fluid flow treatment system 800 including two electrode assemblies 802a, 802b (collectively 802) electrically coupled to a high voltage/high frequency transformer 804, and including a pair of current transducers 806a, 806b, according to one illustrated embodiment.

Hot electrodes 808a-808d (only two called out for each electrode assembly 802a, 802b, collectively 808) of the electrode assemblies 802a, 802b are electrically coupled, directly or indirectly, to one output 810 of the high voltage/high frequency transformer 804. In particular, FIG. 8 shows series electrical coupling of the hot electrodes 808 via conductive paths 812a, 812b (only one called out for each called out electrode assembly 802a, 802b, collectively 812).

Ground electrodes 814a-814d (only two called out for each electrode assembly 802a, 802b, collectively 814) of the electrode assemblies 802a, 802b are electrically coupled, directly or indirectly, to another output 816 of the high voltage transformer 804 and a ground reference node 818. In particular, FIG. 8 shows series electrical coupling of the ground electrodes 814 via conductive paths 818a, 818b (only one called out for each called out electrode assembly 802a, 802b, collectively 818).

Dielectric barrier plates 815a, 815b (only one called out for each electrode assembly 802a, 802b, collectively 815) are positioned between each pair of hot and ground electrodes 808, 814, respectively. The dielectric barrier plates 815 are sized, dimensioned and positioned to prevent undesired arcing or shorting between the hot and ground electrodes 808, 814, respectively.

The current transducers 806a, 806b are electrically coupled between the ground electrodes 814 of respective electrode assemblies 802a, 802b, respectively, and the ground node 818. The current transducers 806a, 806b may be part of a fault monitoring or detection subsystem. In this respect, the hot electrodes of both electrode assemblies 802a, 802b are commonly fed, while ground current from electrode assemblies 802a, 802b are sensed along separate respective paths. If a fault occurs, for example a breakage in a dielectric barrier plate 506 (FIG. 5), power will go to the fault. Thus, the current transducer 806a, 806b coupled to the electrode assembly 802a, 802b that experiences the fault will receive substantially all of the current, while the current transducer 806a, 806b coupled to the electrode assembly 802a, 802b that does experience the fault will receive substantially no current. A controller, processor or other component (e.g., microprocessor 140 of FIG. 1) may monitor the state of the current transducer 806a, 806b. The controller, processor or other component may provide an indication of the particular electrode assembly 802a, 802b that is experiencing the fault. Such may be a visual indication via a light (e.g., LED) and/or display (e.g., LCD), an aural indication via a speaker, and/or an electronic notification via an electronic mail message (i.e., email), voicemail message, text message (i.e., Text), Short Message Service message (i.e., SMS message), etc. The controller, processor or other component may additionally, or alternatively, take precautionary action. For example, the controller, processor or other component may automatically shut down operation, stopping the supply of electrical power to the high voltage/high frequency transformers 420 (FIG. 4) in response to detection of a fault or detection of a fault for a period that exceeds a defined duration.

### Operation

While there are a variety of approaches to operating the treatment systems described above, which approaches may depend on a number of factors including the specific application, fluid volumes and particularly the types of contaminants or pollutants to be treated, a number of specific methods are described below. Variations on such, as well as other such methods, will be readily apparent to one of skill in the art based on the teachings herein.

A control subsystem operates a first stage non-thermal plasma generation cell assembly during a first period to produce a first NTP field in a first flow path with a first set of NTP field characteristics.

The control subsystem operates a second stage NTP generation cell assembly during the first period to produce a second NTP field in the first flow path positioned relatively downstream from the first NTP field in the first flow path with a second set of NTP field characteristics. The second set of NTP field characteristics is different from the first set of NTP field characteristics.

Optionally, the control subsystem operates a third stage NTP generation cell assembly during the first period to produce a third NTP field in the first flow path positioned relatively downstream from the first and the second NTP fields in the first flow path with a third set of NTP field characteristics. The third set of NTP field characteristics may be different from the first and the second sets of NTP field characteristics.

Optionally, the control subsystem operates a parallel first stage non-thermal plasma (NTP) generation cell assembly during the first period to produce a parallel first NTP field in a second flow path. The parallel first NTP field may have the same NTP field characteristics as the first set of NTP field characteristics.

Optionally, the control subsystem operates a parallel second stage NTP generation cell assembly during the first period to produce a second NTP field in the second flow path positioned relatively downstream from the parallel first NTP field in the second flow path. The parallel second NTP field may have the same NTP field characteristics as with the second set of NTP field characteristics.

Optionally, the control subsystem operates a parallel third stage NTP generation cell assembly during the first period to produce a third NTP field in the second flow path positioned relatively downstream from the parallel second NTP field in the second flow path. The parallel third NTP field may have the same NTP field characteristics as with the third set of NTP field characteristics.

The first, the second, and the third sets of NTP field characteristics may include one or more of frequency, power density and wave shape. Operating the first NTP generation cell assembly may include operating the first NTP generation cell assembly to produce an NTP field with the first NTP field characteristics set to destroy a first type of VOC, for example reducing the first type VOC to a second, simpler compound. Operating the second NTP generation cell assembly may include operating the second NTP generation cell assembly with the second NTP field characteristics set to destroy a second type of VOC, for example reducing the second type of VOC to a third, simpler compound. Operating the third NTP generation cell assembly may include operating the third NTP generation cell assembly with the third NTP field characteristics set to destroy a third type of VOC, for example reducing the third type of VOC to a simpler compound. Thus, the third VOC may be relatively less complicated than the second VOC, and the second VOC relatively less complicated than the first VOC.

### Automatic NTP Stage Tuning Search

In some instances a suitable frequency may be found for a given compound via mass spectrometry. However, it may be more efficient to determine suitable frequencies using the treatment system via a tuning algorithm.

One method of tuning an NTP stage to a frequency that will have the best destruction removal efficiency (DRE) for the targeted compound of concern includes programming a control subsystem to automatically step through a defined range of frequencies at predetermined frequency intervals or steps. The control system maintains the physical characteristics (e.g., frequency) of the non-thermal plasma for defined periods or holding times. The physical characteristics or parameters of NTP frequency and power are observed or automatically monitored while these same parameters are controlled. The power density in the NTP field is maintained constant while sweeping the frequency from a first frequency limit to a second frequency limit in defined frequency intervals or steps.

An increase in power will occur at one or more frequencies with the same control settings. Alternatively, the power in the NTP field is maintained with a measurably different voltage control signal. Either situation indicates the occurrence of a type of “resonance” of the power put into the NTP field with the compounds in the field, as the compounds react to their natural resonance frequency. At their natural resonance frequency, the compounds more readily absorb the power applied, and dissociate as the compounds pass through the NTP field. The tuning is performed with the particular compounds which are being tuned for passing through the NTP field, since once the compound dissociates, the resulting compounds or elements will no longer demonstrate this characteristic power change.

The range of frequencies might be quite broad, for example when first investigating what is the appropriate frequency. Notably, there may be more than one frequency that displays this effect. When the resonance setting or settings are found using this approach, then mass spectrometry analysis can be performed around the identified frequencies to verify what reactions are occurring. It is possible that in some instances there is a preferred reaction among those detected. Such may be identified, for example, and the system operated at appropriate settings to achieve the desired reaction.

One method for performing such is described directly below. Other methods will be readily apparent based on this description.

The control subsystem operates a first stage NTP generation cell assembly during a first period to produce a first NTP field in a first flow path with a first set of NTP field characteristics. For example, the control subsystem may operate the first stage NTP generation cell assembly to step through a first range of frequencies for the first NTP field.

The control subsystem detects via one or more sensors a characteristic that is indicative of a first stage ratio of at least one characteristic of the first set of NTP field characteristics. The control subsystem detects via one or more sensors at least one operational characteristic of the first stage NTP generation cell assembly. The first stage ratio may be indicative of an interaction of the first NTP field and a number of compounds in a fluid stream that passes through the first NTP field.

The control subsystem identifies at least one frequency of the first NTP field at which the first stage ratio is optimized. For example, the control subsystem may identify at least one frequency of the first NTP field at which a power of the first NTP field increases without a corresponding change in the operational characteristics of the first stage NTP generation cell assembly. Alternatively, the control subsystem identifies at least one frequency of the first NTP field at which a power of the first NTP field is maintained in response to a decrease of a voltage applied by the first stage NTP generation cell assembly.

The control subsystem may then control the operation of the first stage NTP generation cell assembly to oscillate around the identified frequency during at least one subsequent period based at least in part on a feedback indicative of the first stage ratio. For example, the control subsystem may control the operation of the first stage NTP generation cell assembly to oscillate the first NTP field around the identified frequency in a second range, smaller than the first range.

### Automatic NTP Stage Operations

Once a frequency is selected, a modified version of the NTP tuning algorithm is implemented for the selected stage frequency operation. This is needed to keep the NTP field at the most effective setting to compensate for variations in the best frequency due to effects of concentration, temperature, humidity changes and other effects. The algorithm used here will sweep a narrow range of frequencies in small frequency step changes around the frequency found in the above search and using the above power change detection to keep the operational frequency at the best value.

While there are a variety of approaches to operating the treatment systems described above, a specific method is described below. Variations on such, as well as other such methods, will be readily apparent to one of skill in the art based on the teachings herein.

The control subsystem via at least one sensor detects a characteristic that is indicative of a first stage ratio of at least one characteristic of the first set of NTP field characteristics and at least one operational characteristic of the first stage NTP generation cell assembly. The first stage ratio may be indicative of an interaction of the first NTP field and a number of contaminants in a fluid stream that passes through the first NTP field.

Based on such, the control subsystem identifies at least one frequency of the first NTP field at which a power is minimized.

The control subsystem controls the operation of the first stage NTP generation cell assembly to oscillate around the identified frequency during operation based at least in part on a feedback signal indicative of the first stage ratio.

### Example 1

As an example, a treatment system may have a number of parallel flow paths, each with a number of stages arranged in series in the respective flow path. The stage in any given flow path may have the same operational characteristics as the corresponding stage in the parallel flow paths. Thus, a first stage in each of the flow paths may generate a non-thermal plasma with at least approximately the same physical characteristics as each other. Likewise, a second stage in each of the flow paths may generate a non-thermal plasma with at least approximately the same physical characteristics as each other, but different from the physical characteristics of the non-thermal plasma of the first stages.

The first stage(s) in a flow path is tuned to have maximum effectiveness on the most complex compounds which appear in the fluid flow. Thus, the first stage causes those most complex compounds to dissociate to a lesser molecular complexity.

Where the fluid flow is air containing VOCs to be oxidized and reduced, and the VOCs are a mix of compounds that include 1) long chain hydrocarbon compounds and 2) some hydrocarbon compounds that have the carbon atoms in a benzene ring structure with various single and double bonds, the first stages are tuned to break up the benzene ring bonds so the resulting hydrocarbons are converted from carbon ring type aromatic compounds to long chain hydrocarbon compounds. Subsequent stages (e.g., secondary stages) are tuned to maximum effectiveness for the longest chain hydrocarbon compounds or other compounds remaining. Still subsequent stages (e.g., tertiary stages) are tuned to be effective on those compounds still not fully dissociated or treated from previous stages. Non-thermal plasma stages tuned for disassociating hydrocarbon chain compounds are not specific for the number of carbon atoms are in a hydrocarbon chain. Thus, it is not necessary to have different stages dedicated to each carbon atom count in long chain carbon compounds that are the target of the treatment.

### Example 2

A first stage in a flow path is tuned to achieve maximum destruction of as many VOCs as will be oxidized.

Subsequent NTP stages are tuned for maximum effectiveness on those VOCs remaining from the previous stages.

### CONCLUSIONS

The above description of illustrated embodiments, including what is described in the Abstract, is not intended to be exhaustive or to limit the embodiments to the precise forms disclosed. Although specific embodiments of and examples are described herein for illustrative purposes, various equivalent modifications can be made without departing from the spirit and scope of the disclosure, as will be recognized by those skilled in the relevant art. The teachings provided herein of the various embodiments can be applied to other treatment systems, not necessarily the exemplary series and parallel non-thermal plasma treatment systems generally described above.

For instance, the foregoing detailed description has set forth various embodiments of the devices and/or processes via the use of block diagrams, schematics, and examples. Insofar as such block diagrams, schematics, and examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof. In one embodiment, the present subject matter may be implemented via Application Specific Integrated Circuits (ASICs). However, those skilled in the art will recognize that the embodiments disclosed herein, in whole or in part, can be equivalently implemented in standard integrated circuits, as one or more computer programs executed by one or more computers (e.g., as one or more programs running on one or more computer systems), as one or more programs executed by on one or more controllers (e.g., microcontrollers) as one or more programs executed by one or more processors (e.g., microprocessors), as firmware, or as virtually any combination thereof, and that designing the circuitry and/or writing the code for the software and or firmware would be well within the skill of one of ordinary skill in the art in light of the teachings of this disclosure.

When logic is implemented as software and stored in memory, logic or information can be stored on any computer-readable medium for use by or in connection with any processor-related system or method. In the context of this disclosure, a memory is a computer-readable medium that is an electronic, magnetic, optical, or other physical device or means that contains or stores a computer and/or processor program. Logic and/or the information can be embodied in any computer-readable medium for use by or in connection with an instruction execution system, apparatus, or device, such as a computer-based system, processor-containing system, or other system that can fetch the instructions from the instruction execution system, apparatus, or device and execute the instructions associated with logic and/or information.

In the context of this specification, a “computer-readable medium” can be any element that can store the program associated with logic and/or information for use by or in connection with the instruction execution system, apparatus, and/or device. The computer-readable medium can be, for example, but is not limited to, an electronic, magnetic, optical, electromagnetic, infrared, or semiconductor system, apparatus or device. More specific examples (a non-exhaustive list) of the computer readable medium would include the following: a portable computer diskette (magnetic, compact flash card, secure digital, or the like), a random access memory (RAM), a read-only memory (ROM), an erasable programmable read-only memory (EPROM, EEPROM, or Flash memory), a portable compact disc read-only memory (CDROM), digital tape. Note that the computer-readable medium could even be paper or another suitable medium upon which the program associated with logic and/or information is printed, as the program can be electronically captured, via for instance optical scanning of the paper or other medium, then compiled, interpreted or otherwise processed in a suitable manner if necessary, and then stored in memory.

The various embodiments described above can be combined to provide further embodiments. To the extent that they are not inconsistent with the specific teachings and definitions herein, all of the U.S. patents, U.S. patent application publications, U.S. patent applications, foreign patents, foreign patent applications and non-patent publications referred to in this specification and/or listed in the Application Data Sheet, including but not limited to U.S. Pat. No. 8,105,546, and U.S. Provisional Patent Application Ser. No. 61/618,492, filed Mar. 30, 2012, are incorporated herein by reference, in their entirety. Aspects of the embodiments can be modified, if necessary, to employ systems, circuits and concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An apparatus to treat fluid streams, comprising:
a first stage non-thermal plasma (NTP) generation cell assembly, the first stage NTP generation cell assembly operable to produce a first NTP field in a first flow path;
a second stage NTP generation cell assembly, the second stage NTP generation cell assembly operable to produce a second NTP field in the first flow path positioned relatively downstream from the first NTP field in the first flow path; and
a control system coupled to control the first and at least the second NTP generation cell assemblies to respectively produce during a first period: the first NTP field with a first set of NTP field characteristics and the second NTP field with a second set of NTP field characteristics, the second set of NTP field characteristics different than the first set of NTP field characteristics.

2. The apparatus of claim 1 wherein the first and the second sets of NTP field characteristics include frequency, and the control system is operable to control the first NTP generation cell assembly such that the first NTP field has a first frequency and is operable to control the second NTP generation cell assembly such that the second NTP field has a second frequency, the second frequency different than the first frequency.

3. The apparatus of claim 1 wherein the first and the second sets of NTP field characteristics include at least one of a wave shape or a power level, and the control system is operable to control the first stage NTP generation cell assembly to have at least one of a first wave shape or a first power level and is operable to control the second stage NTP generation cell assembly to have at least one of a second wave shape or a second power level, different than the first wave shape or first power level, respectively.

4. The apparatus of claim 1 wherein the first and the second sets of NTP field characteristics include frequency, wave shape and power level, and the control system is configured to control the first NTP generation cell assembly such that the first NTP field characteristics transforms a first compound to a second compound and to control the second NTP generation cell assembly such that the second NTP field characteristics transforms the second compound to a third compound.

5. The apparatus of claim 1 wherein the first and the second sets of NTP field characteristics include frequency, wave shape and power level, and the control system is configured to control the first NTP generation cell assembly such that the first NTP field characteristics destroy a first volatile organic compound (VOC) and to control the second NTP generation cell assembly such that the second NTP field characteristics destroy a second VOC, the second VOC relatively less complicated than the first VOC.

6. The apparatus of claim 1 wherein the first and the second sets of NTP field characteristics include frequency, wave shape and power level, and the control system is configured to control the first NTP generation cell assembly such that the first NTP field characteristics destroy a first number of types of volatile organic compounds (VOCs) and to control the second NTP generation cell assembly such that the second NTP field characteristics destroy a second number of types of VOCs, the second number of types of VOCs being less than the first number of types of VOCs.

7. The apparatus of claim 1, further comprising:
at least one sensor positioned to detect at least one characteristic that is indicative of a first stage ratio between at least one of the first sets of NTP field characteristics and at least one operational characteristic of the first stage NTP generation cell assembly, where the first stage ratio is indicative of an interaction of the first NTP field and a number of contaminants in a fluid stream that passes through the first NTP field.

8. The apparatus of claim 7 wherein the control system is configured to identify at least one frequency of the first NTP field at which a power is minimized, and to control the operation the first stage NTP generation cell assembly to oscillate around the identified frequency during operation based at least in part on a feedback indicative of the first stage ratio.

9. The apparatus of claim 1, further comprising:
a third stage NTP generation cell assembly, the third stage NTP generation cell assembly operable to produce a third NTP field in the first flow path positioned relatively downstream from the first and the second NTP fields in the first flow path, and
wherein the control system is coupled to control the at least the third NTP generation cell assembly to produce during the first period, the third NTP field with a third set of NTP field characteristics, the third set of NTP field characteristics different than the first and the second sets of NTP field characteristics.

10. The apparatus of claim 9 wherein the first, the second, and the third sets of NTP field characteristics include frequency, wave shape and power level, and the control system is configured to control the first NTP generation cell assembly such that the first NTP field characteristics destroy a first volatile organic compound (VOC), to control the second NTP generation cell assembly such that the second NTP field characteristics destroy a second VOC, and to control the third NTP generation cell assembly such that the third NTP field characteristics destroy a third VOC, the third VOC relatively less complicated than the second VOC, and the second VOC relatively less complicated than the first VOC.

11. The apparatus of claim 1 wherein the first, the second, and the third sets of NTP field characteristics include frequency, wave shape and power level, and the control system is configured to control the first NTP generation cell assembly such that the first NTP field characteristics destroy a first number of types of volatile organic compounds (VOCs), to control the second NTP generation cell assembly such that the second NTP field characteristics destroy a second number of types of VOCs, and to control the third NTP generation cell assembly such that the third field characteristics destroy a third of types of VOCs, the second number of types of VOCs being less than the first number of types of VOCs, and the third number of types of VOCs being less than the second number of types of VOCs.

12. The apparatus of claim 1, further comprising:
a parallel first stage non-thermal plasma (NTP) generation cell assembly, the parallel first stage NTP generation cell assembly operable to produce a parallel first NTP field in a second flow path; and
a parallel second stage NTP generation cell assembly, the parallel second stage NTP generation cell assembly operable to produce a parallel second NTP field in the second flow path positioned relatively downstream from the parallel first NTP field in the second flow path, and
wherein the control system is coupled to control the parallel first and at least the parallel second NTP generation cell assembly to respectively produce during the first period: the parallel first NTP field with the first set of NTP field characteristics and the parallel second NTP field with the second set of NTP field characteristics.

13. The apparatus of claim 1, further comprising:
a gas inlet positioned relatively upstream of the first stage NTP generation cell assembly;
a gas outlet positioned relatively downstream of the second stage NTP generation cell assembly, wherein the first flow path extends between the gas inlet and the gas outlet; and
at least a respective high voltage/high frequency transformer for each of the first and the second stage NTP generation cell assemblies.

14. The apparatus of claim 1 wherein the first stage and the second stage NTP generation cell assemblies are each planar dielectric barrier discharge (DBD) type NTP generation cell assemblies which respectively include at least one electrically hot electrode and at least two electrically ground electrodes provided in an alternating arrangement, and at least one dielectric barrier spaced between the at least one electrically hot electrode and the at least two electrically ground electrodes to provide at least one gap therebetween, and wherein the at least one gap between the at least one electrically hot electrode and the at least two electrically ground electrodes of each of the first stage and the second stage NTP generation cell assemblies form part of the first flow path.

15. The apparatus of claim 14 wherein at least one of the at least one electrically hot electrode and the at least two electrically ground electrodes of each of the first stage and the second stage NTP generation cell assemblies are made of the catalytically active material which is exposed in the first flow path to a fluid stream to be treated during operation of the apparatus and wherein dielectric barriers are one of coated with a catalytically active material or comprised of a catalytically active material.

16. A method of operating an apparatus to treat fluid streams, comprising:
operating a first stage non-thermal plasma (NTP) generation cell assembly during a first period to produce a first NTP field in a first flow path with a first set of NTP field characteristics; and
operating a second stage NTP generation cell assembly during the first period to produce a second NTP field in the first flow path positioned relatively downstream from the first NTP field in the first flow path with a second set of NTP field characteristics, the second set of NTP field characteristics different than the first set of NTP field characteristics.

17. The method of claim 16 wherein the first and the second sets of NTP field characteristics include frequency, and operating the first NTP generation cell assembly includes operating the first NTP generation cell assembly such that the first NTP field has a first frequency and operating the second NTP generation cell assembly includes operating the second NTP generation cell assembly such that the second NTP field has a second frequency, the second frequency different than the first frequency.

18. The method of claim 16 wherein the first and the second sets of NTP field characteristics include at least one of a wave shape or a power level, and operating the first stage NTP generation cell assembly includes operating the first stage NTP generation cell assembly to have at least one of a first wave shape or a first power level and operating the second stage NTP generation cell assembly includes operating the second stage NTP generation cell assembly to have at least one of a second wave shape or a second power level, different than the first wave shape or first power level, respectively.

19. The method of claim 16 wherein the first and the second sets of NTP field characteristics include frequency, wave shape and power level, and operating the first NTP generation cell assembly includes operating the first NTP generation cell assembly such that the first NTP field characteristics transforms a first compound to a second compound and to control the second NTP generation cell assembly such that the second NTP field characteristics transforms the second compound to a third compound.

20. The method of claim 16 wherein the first and the second sets of NTP field characteristics include frequency, wave shape and power level, and operating the first NTP generation cell assembly includes operating the first NTP generation cell assembly such that the first NTP field characteristics destroy a first volatile organic compound (VOC) and operating the second NTP generation cell assembly includes operating the second NTP generation cell assembly such that the second NTP field characteristics destroy a second VOC, the second VOC relatively less complicated than the first VOC.

21. The method of claim 16 wherein the first and the second sets of NTP field characteristics include frequency, wave shape and power level, and operating the first NTP generation cell assembly includes operating the first NTP generation cell assembly such that the first NTP field characteristics destroy a first number of types of volatile organic compounds (VOCs) and operating the second NTP generation cell assembly includes operating the second NTP generation cell assembly such that the second NTP field characteristics destroy a second number of types of VOCs, the second number of types of VOCs being less than the first number of types of VOCs.

22. The method of claim 16, further comprising:
detecting a characteristic that is indicative of a first stage ratio of at least one characteristic of the first set of NTP field characteristics and at least one operational characteristic of the first stage NTP generation cell assembly, where the first stage ratio is indicative of an interaction of the first NTP field and a number of contaminants in a fluid stream that passes through the first NTP field;
identifying at least one frequency of the first NTP field at which a power is minimized; and
controlling the operation the first stage NTP generation cell assembly to oscillate around the identified frequency during operation based at least in part on a feedback signal indicative of the first stage ratio.

23. The method of claim 16, further comprising:
operating a third stage NTP generation cell assembly during the first period to produce a third NTP field in the first flow path positioned relatively downstream from the first and the second NTP fields in the first flow path with a third set of NTP field characteristics, the third set of NTP field characteristics different than the first and the second sets of NTP field characteristics.

24. The method of claim 23 wherein the first, the second, and the third sets of NTP field characteristics include frequency, wave shape and power level, and operating the first NTP generation cell assembly includes operating the first NTP generation cell assembly such that the first NTP field characteristics destroy a first volatile organic compound (VOC), operating the second NTP generation cell assembly includes operating the second NTP generation cell assembly such that the second NTP field characteristics destroy a second VOC, and operating the third NTP generation cell assembly includes operating the third NTP generation cell assembly such that the third NTP field characteristics destroy a third VOC, the third VOC relatively less complicated than the second VOC, and the second VOC relatively less complicated than the first VOC.

25. The method of claim 16 wherein the first, the second, and the third sets of NTP field characteristics include frequency, wave shape and power level, and operating the first NTP generation cell assembly includes operating the first NTP generation cell assembly such that the first NTP field characteristics destroy a first number of types of volatile organic compounds (VOCs), operating the second NTP generation cell assembly includes operating the second NTP generation cell assembly such that the second NTP field characteristics destroy a second number of types of VOCs, and operating the third NTP generation cell assembly includes operating the third NTP generation cell assembly such that the third field characteristics destroy a third of types of VOCs, the second number of types of VOCs being less than the first number of types of VOCs, and the third number of types of VOCs being less than the second number of types of VOCs.

26. The method of claim 16, further comprising:
operating a parallel first stage non-thermal plasma (NTP) generation cell assembly during the first period to produce a parallel first NTP field in a second flow path with the first set of NTP field characteristics; and
operating a parallel second stage NTP generation cell assembly during the first period to produce a second NTP field in the second flow path positioned relatively downstream from the parallel first NTP field in the second flow path with the second set of NTP field characteristics.

27. A method of operating an apparatus to treat fluid streams, comprising:
operating a first stage non-thermal plasma (NTP) generation cell assembly during a first period to produce a first NTP field in a first flow path with a first set of NTP field characteristics;
detecting a characteristic that is indicative of a first stage ratio of at least one characteristic of the first set of NTP field characteristics and at least one operational characteristic of the first stage NTP generation cell assembly, where the first stage ratio is indicative of an interaction of the first NTP field and a number of compounds in a fluid stream that passes through the first NTP field;
identifying at least one frequency of the first NTP field at which the first stage ratio is optimized; and
controlling the operation of the first stage NTP generation cell assembly to oscillate around the identified frequency during at least one subsequent period based at least in part on a feedback indicative of the first stage ratio.

28. The method of claim 27 wherein operating a first stage NTP generation cell assembly during a first period includes operating the first stage NTP generation cell assembly to step through a first range of frequencies for the first NTP field, and controlling the operation of the first stage NTP generation cell assembly to oscillate around the identified frequency during at least one subsequent period includes controlling the operation of the first stage NTP generation cell assembly to oscillate the first NTP field around the identified frequency in a second range, smaller than the first range.

29. The method of claim 27 wherein identifying at least one frequency of the first NTP field at which the first stage ratio is optimized includes identifying at least one frequency of the first NTP field at which a power of the first NTP field increases without a corresponding change in the operational characteristics of the first stage NTP generation cell assembly.

30. The method of claim 27 wherein identifying at least one frequency of the first NTP field at which the first stage ratio is optimized includes identifying at least one frequency of the first NTP field at which a power of the first NTP field is maintained in response to a decrease of a voltage applied by the first stage NTP generation cell assembly.

31. The method of claim 27 wherein the interaction that the first stage ratio is indicative of includes an interaction of the first NTP field and a number of volatile organic compound contaminants in a fluid stream that passes through the first NTP field.
